(19) United States
(12) Patent Application Publication
(10) Pub. No.: US 2010/0268080 A1
(43) Pub. Date: Oct. 21, 2010

(54) APPARATUS AND TECHNIQUE TO INSPECT MUSCLE FUNCTION

(76) Inventor: Larry J. Kirn, Austin, TX (US)

Correspondence Address: GIFFORD, KRASS, SPRINKLE,ANDERSON & CITKOWSKI, P.C, PO BOX 7021, TROY, MI, 48007-7021, US

(21) Appl. No.: 12/760,914

(22) Filed: Apr. 15, 2010

Related U.S. Application Data
(60) Provisional application No. 61/169,511, filed on Apr. 15, 2009.

(51) Int. Cl. A61B 8/00

(52) U.S. Cl. 600/437

(57) ABSTRACT

A system and method for extracting functional and/or diagnostic information from acoustic emissions indicative of muscle activity. Time domain signals are transformed into the frequency domain while preserving temporal content, isolating and preserving the sign of changes of frequencies so obtained in a preserved temporal context, and presenting the frequency derivatives so obtained in the time domain, correlated with other sound features or aspects.

### REFERENCE TO RELATED APPLICATIONS

This application claims priority from U.S. Provisional Patent Application Ser. No. 61/169,511, filed Apr. 15, 2009, the entire content of which is incorporated herein by reference.

### FIELD OF THE INVENTION

This invention relates generally to electronic signal processing and, in particular, to methods and apparatus for acquisition and analysis of acoustic emissions from muscle tissue.

### BACKGROUND OF THE INVENTION

It has been known for quite some time that electrical motor nerve impulses directly result in muscle contraction. It also has been known since the early 19th century that muscles emit sound when contracting. A high degree of correlation has been found between the amplitude of this low-frequency sound and the force exerted by the muscle. In contrast to electromyographic signals which expose nerve stimulation events, acoustic myographic signals provide the physical response of muscle tissue to this stimulation.

Further analysis of specific sound characteristics as they relate to muscle function has been limited, being hampered by the low frequencies involved and myriad noise sources in this spectral range. Although the vast majority of work has concentrated on use of the relatively unqualified amplitude of this sound, some research has inspected spectral components, primarily using fast Fourier transforms (FFTs). As current culmination to this research, several studies have used cepstrum processing, presumably due to its popularity in other sound research. Cepstrum processing is based on use of a spectral transform upon a spectral transform of a signal in the time domain (transform of a transform). Resultantly, it shows depth of frequency agility or movement of specific frequencies, and has been notably beneficial in room and building acoustics.

Although cepstrum processing condenses a great deal of data into a useful form, it does so at the expense of detail. Specifically, the sign of frequency deviations is lost by the process itself, being lumped into an average. Furthermore, FFTs, which are used in the overwhelming majority of cepstrum work, destroy temporal information of the incoming data. By the use of two FFTs, the temporal position in incoming data of both specific frequencies and their movement is made unavailable. This loss is not deleterious with data lacking spectral markers, such as a concert hall design, but fully hides them if these markers exist.

Motor nerve signals are impulse events, or firings. The sound emitted from the muscle correlates to these impulse events, imparting the atonal (noise) characteristic of acoustic myography signals. The physical impulse events, however, are filtered by their travel through variable tissue/fluid media from the source to any means used to capture them. Most of the transmission path is static, but the initial portion of this mechanical filter is the muscle tissue itself. In that the muscle is being contracted, its physical compliance is dynamically decreased. Furthermore, this compliance instantaneously changes in the time frame of individual firings.

The speed of sound through a medium is inversely proportional to the compliance of the medium. Sound travels faster through taut tissue than through flaccid tissue. Resultantly, the filtering frequency taut tissue has on impulse events is higher than the frequency imposed by flaccid tissue.

Increases of specific frequencies at contraction and decreases of specific frequencies are therefore visible in acoustic myography signals, both on a long-term basis of full muscle contraction, and on a short-term basis at each specific firing impulse event. The magnitude of frequency increase correlates to the rate of muscle response, in the time frame being observed. This rate of muscle response is known to be a factor of cell composition (“fast twitch/slow twitch”), oxygenation, fatigue, and many other static and dynamic determinants. The rate of muscle relaxation is as well known to rely upon these and other factors such as cholinergic residual response.

Not only is this information available in acoustic myography signals helpful for transient situations, such as fatigue detection; it shows promise to provide predictive information through more static conditions, such as impending cardiac events. A need exists for a method whereby temporal response of muscle tissue to excitation is measured, analyzed, and depicted.

### SUMMARY OF THE INVENTION

This invention resides in a system and method for extracting functional and/or diagnostic information from acoustic emissions indicative of muscle activity. A system-level implementation includes a transducer for converting acoustic impulses from a muscle into a corresponding electrical signal having temporal and amplitude information, and signal processing circuitry and apparatus operative to continuously or sequentially convert the signal into a frequency-domain signal that preserves the temporal and amplitude information, and represent static or derivative information of individual spectral components within the frequency-domain signal along one or more depiction axes.

In the preferred embodiment the transducer is a microphone such as a piezo film unit. The electrical signal may contain one or more analog or digital constituents, and the conversion to the frequency domain may be at least partially performed in the analog domain or processed digitally using a digital signal processor. The representation of static or derivative information of individual spectral components may be carried out with a visual display. The temporal representation of deviations of individual spectral components may be normalized to a heartbeat or other single muscle event, and a memory may be included for storing information output representations for future comparative use.

A basic method of extracting functional and/or diagnostic information from acoustic emissions representative of muscle activity, comprising the steps of:

1) converting acoustic impulses from a muscle into a corresponding electrical signal having time-domain information;

2) transforming the time-domain information into temporally-accurate frequency-domain spectral components;

3) determining deviations individual spectral components; and of the frequency-domain output information representative of the deviations.

### BRIEF DESCRIPTION OF THE DRAWINGS

FIG. 1 shows a system which presents output as amplitudes and deviations of specific spectral areas; and

FIG. 2 is a block diagram of an embodiment of the invention.

### DETAILED DESCRIPTION OF THE DRAWINGS

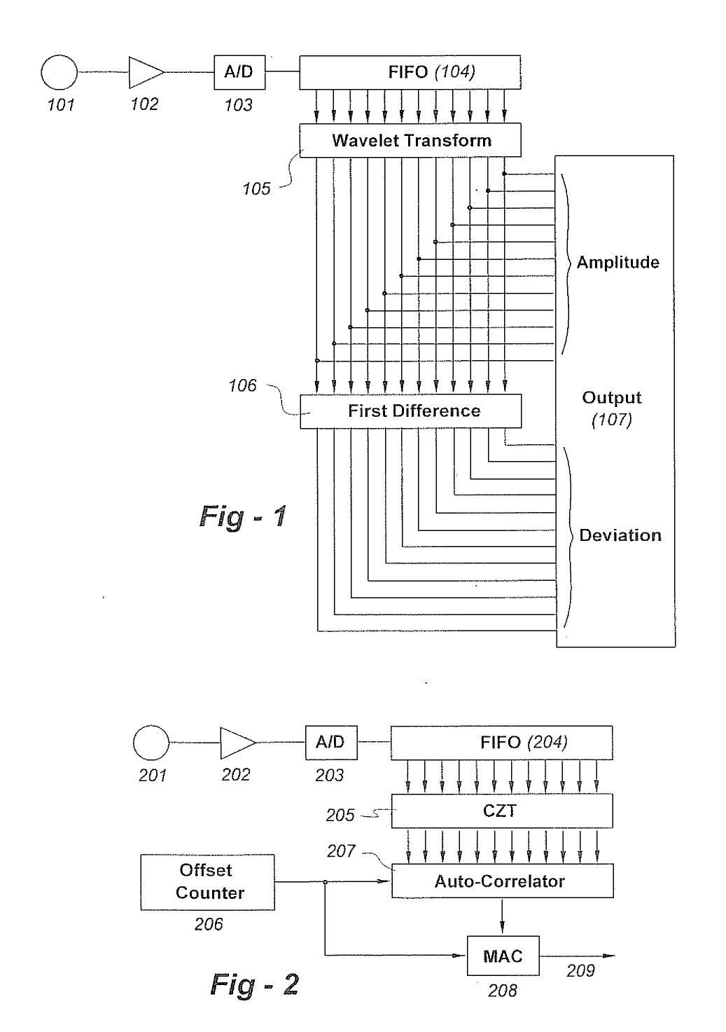

FIG. 1 shows a block diagram of a preferred embodiment of the present invention which presents output as amplitudes and deviations of specific spectral areas. FIG. 2 shows a block diagram of a preferred embodiment of the present invention which presents output as deviation of features in a predetermined spectral area.

Referring now to FIG. 1, Microphone 101 receives acoustic information from a muscle to be inspected. Amplifier 102 conditions this acoustic muscle signal for conversion to digital samples by Analog-to-Digital Converter 103. The samples of acoustic muscle sound are input and sequentially stored in First In First Out Memory 104. Sequentially-stored samples from FIFO 104 are then supplied as input to Wavelet Transform 105. The output of Wavelet Transform 105, consisting of amplitude values for predefined spectral categories, is supplied as input to Output Device 107 and to First Difference Calculation 106. The output of First Difference Calculation 106 is as well supplied to Output Device 107.

The technique of sampling an analog signal, maintaining a signal history, and transforming this time-domain signal to a frequency-domain signal, culminating at Wavelet Transform 105, is well known in the art. In contrast to conventional use of a FFT or DFT to change domains, however, a wavelet transform, which is a member of a transform class which maintains temporal integrity, is used. This integrity is essential to the present invention. The individual amplitude values of each spectral category are provided to the output device.

Common practice in the art is to depict amplitude on one axis (usually Y) as a function of spectral category on the other axis (usually X). Deviation of specific frequencies, however, are implicit only in this amplitude output. Deviation signals from First Difference Calculation 106 are as well provided to the output as a function of spectral category, thus being temporally and spectrally correlated. Common practice in the art is to accentuate data of type by depiction on a third axis (Z), or preferably by modulating the color of the X/Y display by its value. The inclusion of doubly-correlated derivative information to the output fully discloses instantaneous muscle response over a broad range of frequencies.

Referring now to FIG. 2, Microphone 201 receives acoustic information from a muscle to be inspected. Amplifier 202 conditions this acoustic muscle signal for conversion to digital samples by Analog-to-Digital Converter 203. The samples of acoustic muscle sound are input and sequentially stored in First In First Out Memory 204. Sequentially-stored samples from FIFO 204 are then supplied as input to Chirp Transform 205. The output of Chirp Transform 205 is supplied as input to Auto-Correlator 207, which self-correlates amplitude of the incoming spectral components at a temporal offset determined by Offset Counter 206 Offset Counter 206 presumably continuously counts in a positive direction, hence sweeping offset from negative to positive offset, relative to the auto-correlator mid-point. The output of Auto-Correlator 207 and the output of Offset Counter 206 are multiplied and accumulated within a sweep cycle of Offset Counter 206 by MAC Unit 208. The accumulated product from MAC Unit 208 is supplied as Output 209.

It is again of note that the transform used to traverse domains from time to frequency does not destroy temporal information. It is as well helpful, but not fundamental, that a chirp transform requires minimal sample history and processor execution time to yield high-quality results within a limited spectrum.

Location of features within a sample stream using correlation is well-known in the art. Difference calculations through self-correlation resultantly are in broad use. It is presumed that Offset Counter 206 sweeps through its entire range of offsets, from negative through positive, once for each incoming sample period and increment of FIFO 204. Resultantly, any static spectral pattern supplied to Auto-Correlator 207 by CZT 205 will provide maximum correlation output when Offset Counter 206 indicates an offset of zero. The accumulated product of correlation (from Auto-Correlator 207) and offset (from Offset Counter 206) throughout a sweep cycle with a static input will then approach zero. If, however, a spectral feature from CZT 205 changes relative position, the subsequent maximum correlation output from Auto-Correlator 207 will occur at an offset other than zero, due to the phase difference. This offset will be negative or positive, depending on the relative direction of the feature movement from the mid-point of Auto-Correlator 207. The resultant accumulated product at Output 209 after such a feature movement will therefore be negative or positive, at a magnitude corresponding primarily to the offset magnitude. This embodiment then provides a scalar output indicating derivative of frequencies within the transform range of CZT 205, with greater sensitivity but less frequency range then the embodiment of FIG. 1.

By the disclosure and exemplary embodiments herein, an algorithmic approach and apparatus to directly inspect muscle response to activation is seen. Due to the nature of the central principle shown, the embodiments given are but a minor subset of those possible.

1. A system for extracting functional and/or diagnostic information from acoustic emissions indicative of muscle activity, comprising:
a transducer for converting acoustic impulses from a muscle into a corresponding electrical signal having temporal and amplitude information; and
signal processing circuitry and apparatus operative to:
(a) continuously or sequentially convert the signal into a frequency-domain signal that preserves the temporal and amplitude information, and
(b) represent static or derivative information of individual spectral components within the frequency-domain signal along one or more depiction axes.

2. The system of claim 1, wherein the transducer is a microphone.

3. The system of claim 1, wherein the transducer is a piezo film microphone.

4. The system of claim 1, wherein the electrical signal contains one or more analog constituents.

5. The system of claim 1, wherein the electrical signal contains one or more digital constituents.

6. The system of claim 1, wherein the conversion to the frequency domain is at least partially performed in the analog domain.

7. The system of claim 1, wherein the signal processing circuitry includes a digital signal processor.

8. The system of claim 1, wherein the representation of static or derivative information of individual spectral components is accomplished with a visual display.

9. The system of claim 1, wherein temporal representation of deviations of individual spectral components is normalized to a heartbeat or other single muscle event.

10. The system of claim 1, including a memory for storing information output representations for future comparative use.

11. A method of extracting functional and/or diagnostic information from acoustic emissions representative of muscle activity, comprising the steps of:
converting acoustic impulses from a muscle into a corresponding electrical signal having time-domain information;
transforming the time-domain information into temporally-accurate frequency-domain spectral components;
determining deviations individual spectral components; and of the frequency-domain output information representative of the deviations.

12. The method of claim 11, including the step of preserving the sign of the deviations of the individual components.

13. The method of claim 11, wherein the step of transforming the time-domain information is accomplished through chirp or wavelet transformation.

14. The method of claim 11, wherein multiple domain transformations are sequentially performed to reveal deviations of specific frequency components in time.

15. The method of claim 11, wherein the output information is in two or more dimensions.

16. The method of claim 11, wherein the output information includes one depiction axis representative of the deviations in absolute time.

17. The method of claim 11, wherein the spectral deviation of heart beats or other multiple muscle events is combined through statistical averaging.

18. The method of claim 11, wherein the output information includes one depiction axis representative of a heartbeat or other single muscle event.

19. The method of claim 11, wherein the output information includes one depiction axis representative of heart rate, breathing rate or other independent variable.
